(19) United States
(12) Patent Application Publication
(10) Pub. No.: US 2004/0166487 A1
(43) Pub. Date: Aug. 26, 2004

(54) Automated rinse water and body fluid bioagent detection

(76) Inventors: Brian M. Sullivan, Manhattan Beach, CA (US); Denes L. Zsolnay, Rolling Hills Estates, CA (US)

Correspondence Address: RONALD M. GOLDMAN, ROTH & GOLDMAN, SUITE 500, 21535 HAWTHORNE BLVD., TORRANCE, CA, 90503, US

(21) Appl. No.: 10/373,942

(22) Filed: Feb. 25, 2003

(51) Int. Cl. C12Q 1/70; C12Q 1/04

(52) U.S. Cl. 435/5; 435/34

(57) ABSTRACT

Testing of the foot soldier or other person on the battlefield for bioagent contamination is facilitated by a computer controlled portable testing unit that combines a sink or other receptacle (1), an automated ELISA tester (3), and means to transport fluids (10, 9, 11, 15) stemming from the soldier deposited in the receptacle to the automated ELISA tester in which the analysis is performed. For external contamination detection, operation is initiated by a sensor (25) detecting the presence of a person's hands under a spout (23) and for internal contamination detection operation is initiated by the operation of a momentary operate switch (32). External contamination detection begins by washing the soldier's hands and/or face in receptacle (1), while internal contamination detection begins with the soldier spitting, coughing or sneezing into the receptacle.

### REFERENCE TO RELATED APPLICATIONS

Reference is made to U.S. application, Ser. No. 09/837,946, filed Apr. 19, 2001, entitled “Automated Computer Controlled Reporter Device for Conducting Immunoassay and Molecular Biology Procedures,” to U.S. application Ser. No. 09/838,625, filed Apr. 19, 2001, entitled Insect Collection and Test, to U.S. application, Ser. No. 10/055318, filed Oct. 23, 2001, entitled Combinational Strategy for Identification of Biological Agents, to U.S. application Ser. No. 10/116348, filed Apr. 4, 2002, entitled “Combinational Biosensor,” and Docket No. 15-0236, entitled “Charged BioMolecule/Binding Agent Conjugate For Biological Capture,” filed concurrently herewith, all of which are assigned to the assignee of the present application.

### FIELD OF THE INVENTION

This invention relates to early detection of bioagents that might infect soldiers and other individuals, and, more particularly, to non-invasive automatic detection and identification in the field of bioagents through examination of body and wash fluids donated by the soldiers without the need for hospital or laboratory facilities.

### BACKGROUND

To wage war, the nation requires soldiers who are physically fit and capable of using the weapons of war on a battlefield to fight the enemy. To assure fighting capability, much effort is made to recruit or conscript persons who are physically fit to serve as soldiers. Following induction into the service and thereafter effort continues to maintain the soldier's health and fitness on the battlefield, where the soldier's effectiveness is most important.

War has always been a nasty business directed to the subjugation of an enemy. And a successful subjugation typically involves killing or maiming large numbers of enemy soldiers. Conversely, the enemy does all that it can do to kill or maim the nation's soldiers in an effort to avoid being subjugated. Locked in that battle, the credo is to kill or be killed.

Modern technology has enhanced the soldier's ability to kill and maim persons in many ways. That technology includes some weapons that the governments of many nations find insidious and immoral, weapons referred to as chemical and biological weapons, collectively referred to herein as bioagents, capable of producing mass destruction. Released upon the soldiers, those bioagents are able to permanently destroy the health of soldiers surreptitiously, and, render those soldiers exposed to the bioagent incapable of carrying on the battle, often resulting in agonizing death or a life long disability.

Many nations have agreed that bioagent weapons would not be asserted against an enemy in battle. Unfortunately, not all nations adhere to that standard of battlefield morality. The latter countries may be perceived as uncivilized or renegades, ready, willing and able to use those scurrilous weapons. Typically, those renegade nations are lead by malevolent dictators or tyrants. As prime example, Iraq is a nation led by a dictator. That nation has used chemical and biological weapons in a war carried on with Iran and has used those weapons even on groups of its own citizens. So notorious was that nation's use, the coalition of nations that took up arms against Iraq in the Persian Gulf War in 1991 harbored legitimate concern that the army of Iraq would unleash bioagents upon the coalition troops. As a consequence, the troop encampments were monitored for some bioagents, the soldiers were issued protective body gear, and the soldiers were vaccinated or treated for exposure or potential exposure to suspected bioagents.

Unfortunately, the treatments and vaccines for such bioagents were relatively new, even regarded by some as experimental. They did not have a history of safety. With hindsight, the treatments and vaccines given the troops appear to have been unnecessary. Despite good intentions, those unproven vaccines may have been responsible for causing more harm to the soldiers than good through unwelcome and unwanted deleterious side effects. According to one report of the United States Senate, the treatment of the soldiers with experimental drugs or vaccines is the prime suspect in causing “Gulf War Syndrome,” a mysterious illness involving memory loss, thyroid disorders, allergies, fatigue, rashes and persistent pain that afflicts 100,000 veterans who served in the Persian Gulf War in 1991.

As one can now appreciate, to minimize risk to the soldier's health in the foregoing instance the better practice would have been to apply decontamination treatment promptly only after the presence of a bioagent is confirmed and identified. So doing reduces the need for unnecessary medical treatment and exposure to the unwanted side effects of the decontamination agents. While such a procedure may not have been possible at the time of the Persian Gulf War, advancements in technology make such a practice possible today. As additional background that detection technology is next explored.

One accepted procedure or process for detection of a specific bioagent, applicable to a variety of fields, such as biotechnology, environmental protection and public health, is the enzyme linked immunoassay (hereafter referred to as “ELISA”). The ELISA process constitutes an identification process that uses molecular interactions to uniquely identify target substances. A basic definition of ELISA is a quantitative in vitro test for an antibody or antigen (e.g., a bioagent) in which the test material is adsorbed on a surface and exposed to a complex of an enzyme linked to an antibody specific for the substance being tested for with a positive result indicated by a treatment yielding a color in proportion to the amount of antigen or antibody in the test material. The basic ELISA procedure is described more specifically, for one, in a book entitled Methods in Molecular Biology Vol. 42, John R. Crowther, Humana Press, 1995.

The “antibody specific for the substance being tested for” in the foregoing definition constitutes a recognition molecule, a molecule that is capable of binding to either reactant or product molecules in a structure-restricted manner. That is, the recognition molecule binds to a specific three-dimensional structure of a molecule or to a two-dimensional surface that is electrically charged and/or hydrophobic in a specific surface pattern. It may also be recognized that ELISA-like approaches using other recognition molecules can also be used, such as aptamers, DNA, RNA and molecular imprint polymers.

More recently, the foregoing definition for ELISA has been expanded beyond the colormetric approach, wherein color and color intensity is used as a reporter or indicia of the antigen or antibody, to include a voltametric or amperiometric approach to detection. With the latter, the indicia is the rate of change of voltage or current conductivity in proportion to the amount of antigen or antibody contained in the test material. Patent Cooperation Treaty application PCT/US98/16714, filed Aug. 12, 1998 (International Publication No. WO 99/07870), entitled “Electrochemical Reporter System for Detecting Analytical Immunoassay and Molecular Biology Procedures” (hereafter the “16714 PCT application”), claiming priority of U.S. patent application Ser. Nos. 09/105,538 and 09/105,539”), to which the reader may refer, describes both a colormetric and an electrochemical reporter system for detecting and quantifying enzymes and other bioagents in analytical and clinical applications. The electrochemical reporter system of the 16714 PCT application employs a sensor for detecting voltametric and/or amperiometric signals that are produced in proportion to the concentration of organic (or inorganic) reporter molecules by redox (e.g. reduction-oxidation) recycling at the sensor.

In brief, in the ELISA test, the suspect bioagent is initially placed in a water-based buffer, such as a phosphate buffered saline solution, to form a sample solution. That sample solution is mixed with a quantity of particles, such as beads, the surface of which is coated with an antibody to the suspect bioagent, herein also referred to as a recognition molecule and sometimes as a receptor molecule. The particular antibodies used to coat the beads are known to bind to the bioagent of interest or of concern and is a primary antibody or “1° Ab.” That is, the antibody coating exhibits a chemical “stickiness” that is selective to specific bioagents.

Any bioagent present in the sample solution binds with a non-covalent bond to a respective antibody and thereby becomes attached to a respective one of the beads in the mixture-solution. If the sample solution does not contain a bioagent or if the bioagent that is present in the solution is not one that binds to the selected antibody, then nothing binds to the foregoing antibody. Further processing of the ELISA process then shows nothing.

Assuming the suspect bioagent is present in the sample, the bioagent bonds to the antibody that is coated on the beads. The solution then contains a quantity of bioagent molecules bound respectively to a like quantity of coated beads, a 1° Ab/bioagent combination. The mixture is optionally washed, For example, in a phosphate-buffered saline, and a second antibody, more specifically, an antibody and enzyme linked combination, is then added to the mixture. The second antibody is also one that is known to bind to the suspect bioagent, another recognition molecule. The second antibody may either be one that is monoclonal, e.g. one that binds to only one specific molecule, or polyclonal, e.g. a mixture of different antibodies each of which shares the characteristic of bonding to the target bioagent. The enzyme, is covalently bound to the second antibody and forms a complex that is referred to as a secondary antibody-enzyme conjugate or “2° Ab-enz.” As known by those skilled in the art, an enzyme is a “molecular scissors,” a protein that catalyzes a biological reaction, a reaction that does not occur appreciably in the absence of the enzyme. The enzyme is selected to allow the subsequent production of an electrochemically active reporter.

The 2° Ab-enz binds to the exposed surface of the immobilized bioagent to form an “antibody sandwich” with the bioagent forming the middle layer of that sandwich. The antibody sandwich coated beads are washed again to wash away any excess 2° Ab-enz in the solution that remains unbound.

The beads and the attached antibody sandwich, the 1° Ab/bioagent/2° Ab-enz complex, in the solution are placed over the exposed surface of the redox recycling sensor. The substrate of the foregoing enzyme is added to the solution and the substrate is cleaved by the enzyme to produce an electrochemically active reporter. The substrate of the enzyme, referred to as PAP-GP, is any substance that reacts with an enzyme to modify the substrate. The effect of the enzyme is to separate, cut, the PAP, a para-amino phenol, the electrochemically active reporter, from the GP, an electrochemically inactive substance.

The foregoing chemical reaction is concentrated at the surface of the sensor. The rate of production of the foregoing reporter (PAP) is proportional to the initial concentration of bioagent. The reporter reacts at the surface of the sensor, producing an electrical current through the sensor that varies with time and is proportional to the concentration of the bioagent, referred to as redox recycling. The occurrence of the electric current constitutes a positive indication of the presence of the suspect bioagent in the sample. Analysis of the electric currents produced over an interval of time and comparison of the values of that electric current with existing laboratory standards of known bioagents allows quantification of the concentration of bioagent present in the initial sample.

The electrochemical ELISA procedure and apparatus of the cited 16714 PCT application and the predecessor ELISA procedures appear well suited to practice in a microbiology laboratory by highly skilled personnel who are alert to the details of the test process. Other environments in which such an analysis is desirable, such as the battlefield, do not enjoy either the availability of highly skilled technicians or an adequately equipped laboratory.

The availability of a foolproof, user-friendly test apparatus that is able to analyze a sample and report a meaningful result with minimal human intervention is certainly desirable, and recognizing that need, the present inventors, together with other co-inventors, created an automated test apparatus and procedure, which is described in U.S. patent application Ser. No. 09/837,946, filed Apr. 19, 2001, entitled Automated Computer Controlled Reporter Device for Conducting Immunoassay and Molecular Biology Procedures (hereafter the “946 application”), assigned to the assignee of the present application, the content of which is incorporated herein by reference. The apparatus of the '946 application provides a user friendly stand-alone portable unit that is able to automatically perform an ELISA test which may be operated by persons who are not biologists and who require minimal training. The automated system contains a number of solutions and pumps that are controlled by a programmed computer.

The foregoing system also employs beads of magnetic material that are coated with the recognition molecule and a magnetic positioning device to manipulate and position the coated magnetic beads under control of the computer, such as during the washing steps of the ELISA process, and in positioning the beads at the sensor during redox recycling. The automated test device of the '946 application provides a solution to dispersal of testing units that takes into account the lesser skills of prospective operators.

Each of the foregoing ELISA test procedures, whether manual or automatic, and/or colormetric or amperiometric, searches for a single suspect bioagent, and, if detected, determines the concentration of that bioagent. The identification is essentially a “go” or “no-go” procedure. In one approach to identification, the test procedure is repeated serially using different receptor molecules until the bioagent is identified. If the result of the one test is a “no go,” then a second bioagent is made the suspect and the test is repeated for that second bioagent. The foregoing test procedure may be continued a great many times until either the particular bioagent is detected, one exhausts the supply of known receptor molecules or one exhausts the supply of known bioagents.

Even though the ELISA test is automated, as when employing the test apparatus of the cited '946 application, identification of a bioagent could take a great deal of time to accomplish if testing is accomplished in serial order, particularly if the bioagent turns out to be the least likely one in an extended list of suspect bioagents. One solution for reducing the time to identification is to utilize a bank of the foregoing automatic test apparatus, one test apparatus for each bioagent in the group of possibilities, and carry out the separate ELISA test procedures concurrently. Such an approach requires a great deal of equipment, particularly if one tests for a great number of different bioagents. For example, if one is concerned about fifteen different bioagents as possibilities, it is possible to concurrently test using a bank of fifteen automated ELISA testers or other test apparatus, each catered to a respective bioagent.

A more practical approach for bioagent identification is presented in a prior U.S. application, Ser. No. 10/055318, filed Oct. 23, 2001, entitled Combinational Strategy for Identification of Biological Agents (hereafter the “'318 application”), naming one of the present inventors, the content of which is incorporated herein by reference. That invention explicitly recognizes that different recognition molecules (e.g. different types) may be grouped together and used concurrently in an ELISA test to determine if a bioagent is present that links to any one of those different recognition molecules, and, hence, falls within the group of corresponding bioagents. By the invention described in the '318 application, one of up to 2^{N}−1 bioagents in a sample is determined and identified by dividing the sample into N parts and performing N separate identification processes (e.g., ELISA), one process for each of the N parts, N being an integer greater than 1. Each of those N identification processes is assigned a respective group or combination of bioagents to identify. That group is unique to the respective identification process, with the bioagents of the group or combination being selected from the 2^{N}−1 bioagents and with the sum of those bioagents constituting that group or combination being 2^{(N-1)} bioagents, which is less than 2^{N}−1 bioagents.

Each such identification process is capable of identifying any one of a number of bioagents in the group or combination of bioagents assigned for detection to the respective identification process. At least some of the bioagents in the group or combination of bioagents assigned to one identification process are also shared by the group or combination of bioagents that are assigned for identification to at least one other one of the identification processes and each group or combination is assigned a bioagent that is unique to the respective identification process. Each of the N separate identification processes accordingly possess the capability of uniquely identifying a respective single one of the bioagents from the 2^{N}−1 bioagents combination that none of the other identification processes is capable of identifying. By use of combinational logic a particular bioagent is readily identified.

For example, an embodiment in which N equals two, the number of bioagents that can be detected using two ELISA processes is three (i.e. 2²−1). Thus the sample containing one of the bioagents (or none) is parsed in two (i.e. N) parts and each of those parts is separately tested for the bioagents (i.e. N tests). The one ELISA process to test a parsed sample is prepared so that the process is capable of identifying only bioagents A and B; the other ELISA process is prepared to be capable of identifying only bioagents B and C, whereby the identification of bioagent B, common to both processes, is shared. Further, only one of those two processes is uniquely capable of identifying bioagent A, and the other process is uniquely capable of identifying bioagent C. Thus, if both identification processes identify a bioagent, one interpretation is that the bioagent in the sample is B; otherwise, only one of the two identification processes will identify bioagent A or C if present.

Another interpretation is that a combination of bioagents is present, such as both A and C, which is possible, and is an ambiguity. Then another test, such as for either A or C, is taken to resolve any such ambiguity. Essentially in a single major step using two ELISA test apparatus simultaneously, two possible bioagents may be identified at one time and a third may be determined free of any ambiguity with a follow-on test, reducing the necessity for use of three test apparatus or reducing the time for a single test apparatus to perform three separate tests. As the number of bioagents increases, so grows the savings.

As further example, given thirty-one bioagents of interest, only five test apparatus operating concurrently are needed to identify the particular bioagent in a sample, reducing the need for twenty-six test apparatus, concurrently operating, or reducing the time required from that required to perform thirty-one tests with a single test apparatus.

Another similar approach to rapid identification of bioagents is described in U.S. patent application Ser. No. 10/116348, filed Apr. 4, 2002, entitled Combinational Biosensor, one of the present inventors being a co-inventor thereof. The existence of any one of N² bioagents in a sample, in two major steps, N being an integer greater than 1 by first, performing N separate ELISA processes on respective portions of the sample concurrently to identify a group of N bioagents that contain the bioagent, each of the N separate ELISA processes being capable of identifying the presence of a bioagent that falls within a unique group of N bioagents and in which the bioagents detectable by any one of those separate N processes is different from the bioagents that are detectable by any other of the N processes; and then, when a test shows positive, performing N additional ELISA processes on portions of the sample concurrently to identify the particular bioagent; each of the latter N processes being capable of only identifying a respective one of the individual bioagents that form the group of N bioagents identified positive in the previous step.

For example, a total of nine different bioagents may be identified in two series of tests. Using three different test apparatus, each of which is designed to respectively test for a unique group of three bioagents, and running the three tests of a portion of a sample concurrently, the bioagent is first traced to one of the three groups. Then using the three test apparatus, each now programmed to test for a respective one of the three bioagents in that one group, and running the three tests of a portion of the sample concurrently, the individual bioagent may be identified in relatively short order.

The foregoing technology provides a user friendly means to allow relatively unskilled personnel to check for bioagents in the field and to identify the particular bioagent or bioagents. The present invention adapts the foregoing technology to the battlefield.

Accordingly, a principal object of the present invention is to promptly confirm the existence of a bioagent on the battlefield.

A secondary object of the invention is to avoid subjecting soldiers to decontamination agents until the soldier's exposure to a bioagent has been confirmed and the bioagent is identified.

An additional object of the invention is to facilitate operation of the prior automated testing apparatus on the battlefield.

A still additional object of the invention is to equip the automated testing apparatus with an input device to deliver donated fluids for testing.

A still additional object of the invention is to permit the foot soldier to donate specimens for automated analysis in a non-invasive, convenient and natural manner, such as by washing of hands or spitting.

### SUMMARY OF THE INVENTION

The present invention aids the foot soldier, helping the foot soldier avoid the twin evils of suffering from a bioagent attack and adverse unknown reaction due to unnecessary use of vaccines and other decontamination agents. In accordance with the foregoing objects, testing of the foot soldier or other person on the battlefield for bioagent contamination is facilitated by a computer controlled portable testing unit that combines a sink or other fluid receptacle, an automated ELISA tester, and means to transport fluids stemming from the soldier that are deposited in the receptacle to the automated ELISA tester in which the analysis is performed. In accordance with a feature of the invention, operation is initiated by a sensor detecting the presence of the person's hands under a spout or by a person operating a momentary operate switch. External contamination detection begins with the soldier washing the soldier's hands and/or face in the receptacle and being sensed by the sensor. Internal contamination detection begins with the soldier spitting, coughing or sneezing into the receptacle and operating the momentary operate switch. Further in accordance with a feature to the invention, the fluid receptacle comprises a wash-basin and a collection basin

The foregoing and additional objects and advantages of the invention, together with the structure characteristic thereof, which were only briefly summarized in the foregoing passages, will become more apparent to those skilled in the art upon reading the detailed description of a preferred embodiment of the invention, which follows in this specification, taken together with the illustrations thereof presented in the accompanying drawings.

### BRIEF DESCRIPTION OF THE DRAWINGS

In the drawings:

FIG. 1 illustrates an embodiment of the invention; and

FIG. 2 is a timing diagram illustrating operation of certain of the components of the embodiment of FIG. 1 during operation of the embodiment.

### DETAILED DESCRIPTION OF THE PREFERRED EMBODIMENTS

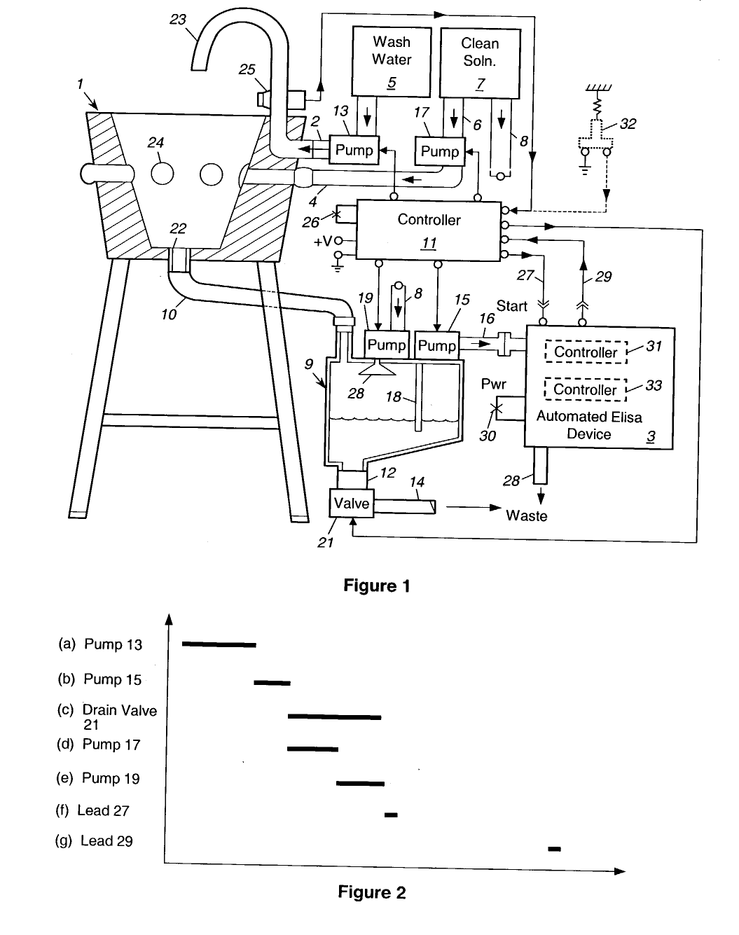

Reference is made to FIG. 1, which illustrates a first embodiment of the detection system. The system includes a receptacle, such as a wash basin 1, an automated bioagent tester 3, a reservoir or container of water which may contain buffers, salts, detergents and/or surfactants 5, a source of a cleaning solution 7, a collection container or basin 9, and an electronic controller 11. The system also includes a number of electrical pumps 13, 15, 17 and 19, an electric stop valve 21, various conduits 2, 4, 6, 8, 10, 12, 14, and 16, and other components, best described herein in connection with the description of the operation of the system.

Wash basin 1 includes a water spout 23, the outlet end of which being directed into the well of the wash basin, a drain 22, a number of spray heads 24 that open into the well of the wash basin from the side walls of the basin, and a sensor 25 to detect the presence of an individual standing in front of the wash basin and the hands of that individual, when the individuals hands are placed under the water spout 23 for washing while standing in front of the wash basin. A source of electrical power, not illustrated, such as a DC battery or engine-generator set, is connected to the +V terminal of the controller 11 to supply the electrical power for operation of the system. Power is applied to the controller by operating start switch 26, wherein the controller initializes for the washing operation, and applies power to sensor 25.

When the soldier places his hands under water spout 23, sensor 25 detects the presence of those hands and signals controller 11. The controller in turn responds by energizing pump 13 for an interval sufficient to permit the soldier enough water to wash his hands and face. That interval is represented as (a) in the timing chart of FIG. 2 to which brief reference is made.

The timing chart of FIG. 2 that identifies the timing interval and sequence of pumps 13, 15, 17 and 19 and valve 21 during the operation of the system. The abscissa represents time and the ordinate represents the individual components that controller 11 energizes (and, hence, which operates) during the respective defined intervals. As illustrated (a) represents the time and interval in which pump 13 is energized and operating; (b) represents the operation of pump 15; (c) the operation of drain valve 21; (d) the operation of pump 17; (e) the operation of pump 19. Thereafter, the controller resets the sequencing operation in preparation for a succeeding wash and test operation.

Being energized, pump 13 pumps water from reservoir 5 through conduit 2 and into an end of water spout 23. The wash water flows out the spout and onto the soldier's hands being washed. The spent wash water drains into the well of the wash basin, and runs out drain 22 at the bottom of the basin. Dispensing of the wash fluid continues for a prescribed interval sufficient to permit the soldier to also wash his face, should the soldier wish. That washing cleans the soldier's skin of any dirt and any bioagents, and the latter, dissolved or in solution with the spent wash fluid, runs out drain 22 under the influence of gravity.

The potentially contaminated wash fluid, which is to be tested for bioagents, is fed from drain 22 into the catch basin 9 via hose 10, connected to drain 22. The spent wash water collects in and partially fills the collection basin 9, rising above the level at the bottom end of the inlet pipe 18 to pump 15. At the conclusion of the washing interval, controller 11 energizes pump 15 for a prescribed interval as depicted in FIG. 2 (b). During that interval, pump 15 draws a quantity of the wash water out of collection basin 9 and pumps that wash water, via coupling hose 16, into the inlet of automated tester 3. Controller 11 then signals the automated test via output line 27 to commence the automated operation that tests the wash water for a bioagent.

Automated ELISA device 3, the automated tester illustrated in the figure, may be a single automated tester or may be formed of a bank of those automated testers. If testing for a single selected bioagent, automated tester 3 may be a testing apparatus described in the prior U.S. Application by the applicants, Ser. No. 09/837,946, filed Apr. 19, 2001, entitled “AUTOMATED COMPUTER CONTROLLED REPORTER DEVICE FOR CONDUCTING IMMUNOASSAY AND MOLECULAR BIOLOGY PROCEDURES” (the “946 application”) or may be that tester described in TRW Docket No. 15-0236, entitled, “CHARGED BIO-MOLECULE/BINDING AGENT CONJUGATE FOR BIOLOGICAL CAPTURE,” the disclosures of which are incorporated herein by reference.

In brief, the automated tester disclosed in the '946 application is an automated computer-controlled tester for performing assessments of immunologic and molecular biology molecules, namely ELISA and ELISA-like assays, in accordance with the steps prescribed by the program of the computer and displays the results obtained on a display 31 for the operator to view. An electronic controller 33 includes a programmed microprocessor (e.g. computer). The foregoing apparatus accepts a sample that may contain a suspect bioagent or the like and automatically treats the sample solution with recognition molecules and reporter molecules in a prescribed sequence to produce an electrical signal at a sensor, automatically inspects the results obtained from the signal over a short interval, processes those results to obtain a number, the slope, that represents the concentration of the suspect bioagent or the like in the solution, and displays the result on a display, such as one of the liquid crystal type, etc., or reports it over some data collection system.

It is recognized that the automated tester of the '946 application is general purpose in application, and, to incorporate that testing apparatus as part of the present invention, some minor modifications are necessary. A fluid conduit is added between front end of the tester that couples into the sample vessel or reservoir, not illustrated, inside the tester. Electrical connections are made internal of automated tester 3 to permit controller 11 to provide a signal via lead 27 to the controller 33 of the tester to start the testing and to provide feedback from the controller 33 of tester 3 to controller 11 via lead 29 to signal to controller 11 that testing is completed.

Alternatively, automated tester 3 may be formed of a bank, not illustrated, of either of the automated testers previously described, in which case coupling hose 16 would be connected in parallel to the inlet of the multiple testers. A bank of the foregoing automated testers may be arranged to accomplish the kind and type of testing described in prior U.S. application, Ser. No. 10/055318, filed Oct. 23, 2001, entitled “COMBINATIONAL STRATEGY FOR IDENTIFICATION OF BIOLOGICAL AGENTS,” and to Ser. No. 10/116348, filed Apr. 4, 2002, entitled “COMBINATIONAL BIOSENSOR,” which are incorporated herein by reference, all of which are assigned to the assignee of the present application. In as much as the detail of the preceding automated testers is not necessary to an understanding of the present invention, it is not necessary to describe the details of that testing apparatus herein with further particularity. The interested reader may refer to those applications for further particulars.

As elsewhere herein described, testing of a specimen takes some time to complete. When completed, the result of the testing is indicated to the operator on a display 31 of the tester 3. While awaiting the test outcome, the controller 11 is programmed to commence the steps to prepare the wash basin 1 and collector basin 9 for use by another soldier. That preparation includes emptying the remaining wash water and cleaning the wash basin and collection basin.

Controller 11 energizes electric valve 21, which opens, allowing the wash water remaining in collection basin 9 to be pumped or to drain by gravity over an interval (see FIG. 2(c)) through drain hose 12, valve 21 and drain hose 14 to a waste or sewer system, not illustrated. While maintaining valve 21 energized, the controller energizes pump 17 for an interval, represented in FIG. 2(d). The pump aspirates neutralizing or cleaning solution from reservoir 7 and pumps the solution into the sprayer channel formed in the wash basin via hose 4. The sprayer channel connects the multiple sprayer heads 24 in the wash basin together. Under the positive pressure produced in the channel by pump 17, the solution is sprayed from the sprayers onto the walls of the wash basin, effectively hosing down the walls of any residual wash water and/or bioagent. The cleaning solution and any dissolved waste empties through the drain 22 into collection basin 9. Since valve 21 remains open, that cleaning solution also drains out of the collection basin as well, passing through hose 12, valve 21, hose 14 to the waste sewer, not illustrated.

On conclusion of the foregoing interval, pump 17 is deenergized, and the controller energizes pump 19 for a succeeding interval as represented in FIG. 2 (e), while maintaining valve 21 open, FIG. 2(c). Pump 19 draws cleaning solution from reservoir 7 via conduit 8 and pumps that solution through sprayer 28, which is located inside collection basin 9. The solution is sprayed about the inside of the collection basin washing down the walls and bottom, and draining through the open valve 12 to the waste sewer.

The foregoing cleans all conduits and basins, except hose 16 leading into the inlet of tester 3. That hose should not contain any significant bioagent residue, and, hence, need not be washed with the cleaning solution like the basins. Should however it be found to be a source of potential residual, then it is possible to adjust the controller so that valve 21 is temporarily closed during the operation of pump 19 to permit temporary accumulation of the cleaning solution pumped through sprayer 28 into the collection basin. Then pump 15 may be reenergized to pump a portion of that cleaning solution through hose 16 into the tester 3, where the solution may be diverted to a waste drain, such as 28, associated with the tester. Thereafter valve 21 may be re-energized to open and drain the spent cleaning solution that remained in collection basin 9.

Following the foregoing cleaning steps, controller 11 resets and remains ready to begin the cycle anew once tester 3 provides a signal to the controller 11, indicating the testing has completed.

As one appreciates, in alternate embodiments, spent water may be pumped directly from wash basin 1 to the test apparatus eliminating a collection basin, such as collection basin 9 and associated cleaning pump 19. However, such an alternative requires that the user place a stopper in drain 22 to block premature draining of the basin, and, should the user use the stopper, remove it before the sink is automatically cleaned. Because the foregoing actions cannot be guaranteed that alternative is less preferred. As a preventive, such an embodiment would require inclusion of an automatic electrically controlled drain valve in wash basin 1, in lieu of a stopper, and for that drain valve to be controlled by controller 11 to close the drain when hand washing is to commence and to open the drain after the test sample has been transferred to tester 3. The latter structure appears to be more complicated to manufacture and less reliable than the configuration of FIG. 1 with the collection basin.

As one appreciates, the foregoing apparatus and the automated ELISA device may be integrated in a single structure or, as preferred and illustrated, as an add-on attachment to the automated ELISA device.

The foregoing embodiment was described in connection with the detection of a bioagent on the skin of a soldier's hands and/or face in which the bioagent is collected in the spent wash water, and a portion of that fluid is pumped into the automated testing device and tested. The foregoing may also be used to check for a bioagent inside the soldier's body. That is, the soldier may be instructed to develop saliva and expectorate into the basin. In that case the saliva fluid merges with the spent wash water. It is also possible for the soldier to cough into basin 1, expelling phlegm into the wash water or to sneeze into the wash water, letting the droplets expelled by the sneeze to enter into the wash water. The testing of the fluid then reports on the bioagent that is located, but does not determine whether the bioagent was found on the soldier's skin or in the soldier's saliva (or phlegm or sneeze droplettes).

If it is necessary to determine if the bioagent derived from the soldier's saliva, the foregoing can be accomplished by the foregoing structure with slight modification as a separate test. Referring to FIG. 1, a momentary operate switch 32 may be installed on the front or side wall of basin 1 and connected in circuit to an input to controller 11, such as the same input used by sensor 25. Operation of the switch is detected by the controller and the controller then runs the program previously described, which need not be repeated, commencing with the pumping of wash water through spout 23 and into receptacle 1.

As one realizes, the foregoing employs a greater volume of wash water than is necessary to wash down the phlegm or the like from basin 1. To conserve wash water, as an alternative, controller 11 may be modified to provide a separate input for momentary switch 32 and to modify the controller operation so that when switch 32 is momentarily closed the period in which pump 13 is energized is reduced to a shorter period (e.g. the programmed time is overridden and replaced by a shorter time). Hence, the volume of wash water pumped through spout 23 is reduced. Like program adjustments in controller 11 may be made as appropriate based on experience with the time of operation of pumps 17 and 19 as well during the cleaning cycle in the foregoing mode of operation.

It is believed that the foregoing description of the preferred embodiments of the invention is sufficient in detail to enable one skilled in the art to make and use the invention without undue experimentation. However, it is expressly understood that the detail of the elements comprising the embodiment presented for the foregoing purpose is not intended to limit the scope of the invention in any way, in as much as equivalents to those elements and other modifications thereof, all of which come within the scope of the invention, will become apparent to those skilled in the art upon reading this specification. Thus, the invention is to be broadly construed within the full scope of the appended claims.

What is claimed is:

1. The method of detecting a bioagent in a fluid that stems from a person, comprising the steps of:
collecting said fluid in a receptacle;
pumping at least a portion of said fluid into an automated bioagent detection apparatus;
activating said automated bioagent detection apparatus to test said portion of said fluid for the presence of said bioagent; and
reviewing the results of said test to determine if said bioagent is present.

2. The method of detecting a bioagent in a fluid that stems from a person as defined in claim 1, wherein said step of collecting said fluid in a receptacle, further comprises:
washing a surface of a human body with wash water to produce spent wash water, said spent wash water collecting any bioagent washed from said surface of said human body; and
depositing said spent wash water in said receptacle.

3. The method of detecting a bioagent in a fluid that stems from a person as defined in claim 1, wherein said step of collecting said fluid in a receptacle, further comprises:
producing saliva; and
expectorating said saliva into said receptacle.

4. The method of detecting a bioagent in a fluid that stems from a person as defined in claim 1, wherein said step of collecting said fluid in a receptacle, further comprises:
producing a sneeze for emitting droplets of fluid from a persons mouth; and
directing said sneeze into said receptacle.

5. The method of detecting a bioagent in a fluid that stems from a person as defined in claim 1, wherein said step of collecting said fluid in a receptacle, further comprises:
producing a cough for emitting fluid from a persons mouth; and
directing said emitted fluid into said receptacle.

6. The method of detecting a bioagent in a fluid that stems from a person as defined in any of claims 3, 4 and 5, further comprising the step of introducing a fluid from an external source into said receptacle to mix with the contents of said receptacle.

7. The method of detecting a bioagent contaminating a surface of a person, comprising the steps of:
washing the surface with wash water to collect any bioagent washed from said surface in the said wash water;
collecting said wash water;
pumping a portion of said collected wash water into an automated bioagent detection apparatus; and
activating said automated bioagent detection apparatus to automatically test said portion of said collected wash water for a bioagent.

8. The method of detecting a bioagent contaminating a surface of a person as defined in claim 7, wherein said automated bioagent detection apparatus automatically tests for any of a variety of different bioagents.

9. Apparatus for detecting the presence of a bioagent on the hands of a person, comprising:
a spout for dispensing wash fluid onto the hands of a person;
a basin for collecting spent wash fluid;
a controller;
an automated bioagent tester for testing spent wash fluid when commanded by said controller;
a first electrical pump for pumping wash fluid through said spout when energized by said controller;
a second electrical pump for pumping a portion of said spent wash fluid into said automated bioagent tester, when energized by said controller.

10. The apparatus for detecting the presence of a bioagent on the hands of a person as defined in claim 9, further comprising:
a sensor for detecting the presence of human hands under said spout, and in response, providing a signal to said controller indicative of such presence.

11. The apparatus for detecting the presence of a bioagent on the hands of a person as defined in claim 9, further comprising:
a sensor for detecting the presence of a human in front of said spout and human hands beneath said spout, and in response thereto, providing a signal to said controller indicative of such presence.

12. The apparatus for detecting the presence of a bioagent on the hands of a person as defined in claim 9, further comprising:
a third electric pump for pumping cleaning solution into said basin when energized by said controller.

13. The apparatus for detecting the presence of a bioagent on the hands of a person as defined in claim 11, further comprising:
a third electric pump for pumping cleaning solution into said basin when energized by said controller.
